(19) United States
(12) Patent Application Publication
(10) Pub. No.: US 2026/0047812 A1
(43) Pub. Date: Feb. 19, 2026

(54) SUPPORT STRUCTURE FOR SUPPORTING AN EXAMINATION OBJECT IN A PEDIATRIC COMPUTED TOMOGRAPHY EXAMINATION AND COMPUTED TOMOGRAPHY DEVICE

(71) Applicant: Siemens Healthineers AG, Forchheim (DE)

(72) Inventors: Martin RAMSAUER, Pyrbaum (DE); Jens FEHRE, Hausen (DE)

(73) Assignee: Siemens Healthineers AG, Forchheim (DE)

(21) Appl. No.: 19/297,050

(22) Filed: Aug. 12, 2025

(30) Foreign Application Priority Data
Aug. 13, 2024 (DE) 10 2024 207 709.7

(51) Int. Cl. A61B 6/04; A61B 6/03

(52) U.S. Cl. CPC A61B 6/0428; A61B 6/032

(57) ABSTRACT

One or more example embodiments relates to a support structure for supporting an examination object in a pediatric computed tomography examination. The support structure includes a bearing surface for the examination object; a first side wall; and a second side wall, wherein the bearing surface, the first side wall and the second side wall in each case extend flat along a longitudinal axis of the support structure, and the first side wall and the second side wall protrude relative to the bearing surface such that the examination object is placeable on the bearing surface between the first side wall and the second side wall.

### CROSS-REFERENCE TO RELATED APPLICATION(S)

The present application claims priority under 35 U.S.C. § 119 to German Patent Application No. 10 2024 207 709.7, filed Aug. 13, 2024, the entire contents of which are incorporated herein by reference.

### FIELD

One or more example embodiments relates to a support structure for supporting an examination object in a pediatric computed tomography examination. One or more example embodiments further relates to a computed tomography device.

### RELATED ART

In computed tomography examinations, precise alignment of the examination object is important for image quality. Herein, in the case of pediatric computed tomography examinations, additional problems may arise, for example with regard to the safety and/or cleanability of the support.

### SUMMARY

One or more example embodiments enables improved supporting of an examination object in a pediatric computed tomography examination. Independent of the grammatical term usage, individuals with male, female or other gender identities are included within the term.

### BRIEF DESCRIPTION OF THE DRAWINGS

The following explains features with reference to examples and with reference to the attached figures. The representation in the figures is schematic, highly simplified and not necessarily true to scale.

FIG. 1 shows a mobile computed tomography device according to one or more example embodiments.

FIG. 2 shows a computed tomography device with an examination table according to one or more example embodiments.

FIG. 3 to FIG. 6 show different views of a support structure according to one or more example embodiments.

FIG. 7 shows a further view of a mobile computed tomography device according to one or more example embodiments.

FIG. 8 shows a cavity for accommodating the holding structure in a form-fitting manner according to one or more example embodiments.

FIG. 9 shows a holding structure in a first orientation according to one or more example embodiments.

FIG. 10 shows an arrangement of a support structure on a gantry according to one or more example embodiments.

FIG. 11 shows a holding structure in a second orientation according to one or more example embodiments.

FIG. 12 shows an arrangement of a support structure on a table top according to one or more example embodiments.

### DETAILED DESCRIPTION

One or more example embodiments relates to a support structure for supporting an examination object in a pediatric computed tomography examination,
wherein the support structure has a bearing surface for the examination object, a first side wall and a second side wall,
wherein the bearing surface, the first side wall and the second side wall in each case extend flat along a longitudinal axis of the support structure,
wherein the first side wall and the second side wall protrude relative to the bearing surface in such a way that the examination object can be placed on the bearing surface between the first side wall and the second side wall, in particular can be placed in such a way that a longitudinal body axis of the examination object is substantially parallel to the longitudinal axis of the support structure.

The examination object can, in particular, be a person. The examination object can, for example, be a baby or a toddler. The examination object can, for example, be supported in a lying position and/or relative to a gantry of a computed tomography device. The computed tomography device can in particular be embodied as a head computed tomography device for a head of an adult and/or as a mobile computed tomography device.

In particular, it can be provided that a depression is embodied between the first side wall and the second side wall in such a way that the examination object can be placed into the depression onto the bearing surface. In particular, the bearing surface can form a base of the depression. In particular, it can be provided that the bearing surface extends flat transversely to the longitudinal axis of the support structure from the first side wall to the second side wall.

In particular, it can be provided that the first side wall and the second side wall protrude relative to the bearing surface in such a way that, due to their planar moment of inertia, they counteract bending of the bearing surface about a transverse axis of the support structure. In particular, it can be provided that the bearing surface extends flat along a transverse axis of the support structure from the first side wall to the second side wall and/or that the transverse axis of the support structure is perpendicular to the longitudinal axis of the support structure.

In particular, it can be provided that the bearing surface is made of carbon fiber-reinforced plastic, for example in such a way that a material thickness of the bearing surface is less than 6 mm, in particular less than 4 mm, in particular not greater than 3 mm. In particular, it can be provided that the first side wall is made of carbon fiber-reinforced plastic, for example in such a way that a material thickness of the first side wall is less than 6 mm, in particular less than 4 mm, in particular not greater than 3 mm. In particular, it can be provided that the second side wall is made of carbon fiber-reinforced plastic, for example in such a way that a material thickness of the second side wall is less than 6 mm, in particular less than 4 mm, in particular not greater than 3 mm.

One embodiment provides that an elongated hole for inserting (or receiving) a first palm of an operator is embodied in the first side wall in such a way that the first palm can enclose an edge region of the first side wall bordering this elongated hole and/or that an elongated hole for inserting (or receiving) a second palm of an operator is embodied in the second side wall in such a way that the second palm can enclose an edge region of the second side wall bordering this elongated hole.

In this way, handles can be embodied that enable easy transportation and easy insertion and/or removal of the support structure at interfaces provided for this purpose.

One embodiment provides that the support structure has a first connecting unit and a second connecting unit for connection, in particular a velcro connection, with a holding strap, wherein the first connecting unit is fastened to the first side wall in such a way and the second connecting unit is fastened to the second side wall in such a way that the first side wall, the holding strap, the second side wall and the bearing surface together form a circumferentially closed contour when the holding strap is connected to the first connecting unit and to the second connecting unit.

In particular, it can be provided that the first connecting unit is fastened to the first side wall in such a way that the first side wall is located between the first connecting unit and the examination object when the examination object lies on the bearing surface between the first side wall and the second side wall and/or that the second connecting unit is fastened to the second side wall in such a way that the second side wall is located between the second connecting unit and the examination object when the examination object lies on the bearing surface between the first side wall and the second side wall.

One embodiment provides that the support structure has a rear wall, wherein the rear wall protrudes relative to the bearing surface and extends flat transversely to the longitudinal axis of the support structure from the first side wall to the second side wall.

In particular, it can be provided that the rear wall protrudes relative to the bearing surface in such a way that, due to its planar moment of inertia, it counteracts bending of the bearing surface about the longitudinal axis of the support structure. Hence, the embodiment of the side walls and the rear wall can enable sufficient stability with the lowest possible material thickness. One embodiment provides that a hole for suspending the support structure, in particular on a hook, is embodied in the rear wall.

One embodiment provides that the bearing surface, the first side wall and the second side wall merge into one another in one piece and/or in the shape of a shell. In particular, it can be provided that the bearing surface, the first side wall, the second side wall and the rear wall merge into one another in one piece and/or in the shape of a shell.

This enables edges that are difficult to clean to be avoided. Furthermore, it can be provided that, at the end of the support structure opposite the rear wall in relation to the longitudinal axis of the support structure, there is no wall protruding relative to the bearing surface. This enables contaminants, for example body fluids from the examination object, to drain from the bearing surface via this end. In the area of the rear wall, the support structure can be embodied as closed over an area in such a way that no liquid contaminants can flow off the bearing surface through the support structure when the bearing surface is arranged substantially horizontally.

One embodiment provides that the support structure has a longitudinal marking, which is arranged on the bearing surface and extends centrally between the first side wall and the second side wall along the longitudinal axis of the support structure. This can support the best possible alignment of the examination object relative to the support structure. The longitudinal marking can, for example, be printed, glued and/or applied to the bearing surface and/or engraved into the bearing surface. The longitudinal marking can in particular contrast with the color of the support structure. For example, the longitudinal marking can be light-colored, in particular white, if the support structure is dark-colored, in particular black.

One or more example embodiments further relates to a computed tomography device having a gantry with an opening and the support structure according to one or more example embodiments,
wherein the opening extends in a tunnel shape along a system axis of the gantry in such a way that the support structure and the examination object can be introduced together along the system axis into the opening when the longitudinal axis of the support structure is parallel to the system axis and the examination object lies on the bearing surface between the first side wall and the second side wall,
wherein the computed tomography device has a first device-side interface,
wherein the support structure has a support-side interface,
wherein the first device-side interface and the support-side interface can establish a first detachable connection, which fixes the support structure relative to the gantry in such a way that the longitudinal axis of the support structure is parallel to the system axis.

In particular, it can be provided that the computed tomography device has an examination table, wherein the examination table has the first device-side interface. For example, the examination table can have a base and a support plate, wherein the support plate is configured to support a person, in particular an adult, in a lying position and is mounted displaceably relative to the base parallel to the system axis of the gantry.

In particular, it can be provided that the first device-side interface is fastened to the support plate in such a way that, upon displacement of the support plate relative to the base, the first device-side interface rests relative to the support plate and/or that the support structure can be inserted into the opening by displacing the support plate relative to the base when the first device-side interface and the support-side interface establish the first detachable connection, which fixes the support structure relative to the gantry in such a way that the longitudinal axis of the support structure is parallel to the system axis. The support-side interface can, for example, have an aluminum housing.

One embodiment provides that the gantry has a first gantry part and a second gantry part, wherein the first gantry part has a projection data acquisition system, wherein the second gantry part has the first device-side interface, wherein the first gantry part is mounted movably relative to the second gantry part in such a way that a translational movement of the first gantry part relative to the second gantry part can be carried out parallel to the system axis, while at the same time the support structure rests relative to the second gantry part, wherein the first device-side interface and the support-side interface establish the first detachable connection, which fixes the support structure relative to the gantry in such a way that the longitudinal axis of the support structure is parallel to the system axis.

One embodiment provides that the support structure has a first transverse marking and a second transverse marking, which are in each case arranged on the bearing surface and extend perpendicular to the longitudinal axis of the support structure, wherein the first transverse marking and the second transverse marking mark an extension along the system axis of an examination area, which is accessible to the projection data acquisition system by the translational movement of the first gantry part relative to the second gantry part.

This can support the best possible alignment of the examination object relative to the support structure. The transverse markings can, for example, be printed, glued and/or applied to the bearing surface and/or engraved into the bearing surface. The transverse markings can in particular contrast with the color of the support structure. For example, the transverse markings can be light-colored, in particular white, when the support structure is dark-colored, in particular black.

One embodiment provides that the second gantry part has a second device-side interface, wherein the second device-side interface and the support-side interface can establish a second detachable connection, which fixes the support structure in such a way relative to the gantry that the longitudinal axis of the support structure is perpendicular and at an angle to the system axis. One embodiment provides that the second gantry part has a chassis, wherein the chassis is configured to move the gantry relative to a floor of an examination room parallel to a translation direction, wherein the translation direction is perpendicular to the system axis.

In particular, it can be provided that the system axis is horizontal and/or that the translation direction is horizontal. In particular, it can be provided that the longitudinal axis of the support structure is substantially parallel to the translation direction when the second device-side interface and the support-side interface establish the second detachable connection, which fixes the support structure relative to the gantry in such a way that the longitudinal axis of the support structure is perpendicular and at an angle to the system axis. This makes it possible to reduce the space required for the computed tomography device transversely to the translation direction for moving the gantry relative to the floor of the examination room.

One embodiment provides that the computed tomography device has a holding structure for the support structure, wherein a first partial structure of the holding structure has the second device-side interface, wherein the second gantry part has a cavity for accommodating the holding structure in a form-fitting manner in such a way that the first partial structure of the holding structure protrudes out of the cavity when the holding structure is accommodated in the cavity in a form-fitting manner.

In particular, it can be provided that a contact surface of the second partial structure of the holding structure rests against a floor of the cavity when the holding structure is accommodated in the cavity in a form-fitting manner. The holding structure can, for example, be made of plastic, in particular a thermoplastic, preferably polyoxymethylene (POM).

One embodiment provides that a second partial structure of the holding structure has a table-side interface, wherein the table-side interface and the support-side interface can establish a third detachable connection, which fixes the support structure relative to the holding structure in such a way that the longitudinal axis of the support structure is substantially parallel to a table top when the holding structure is placed on the table top in such a way that a contact surface of the first partial structure of the holding structure rests against the table top. The contact surface of the first partial structure of the holding structure and the contact surface of the second partial structure of the holding structure can in particular be plane-parallel.

One embodiment provides that the holding structure has a first leg region, a second leg region and a vertex region, wherein the vertex region has the second device-side interface and/or the table-side interface and extends from the first leg region to the second leg region, wherein the first leg region and the second leg region extend parallel to the longitudinal axis of the support structure away from the vertex region when the second detachable connection or the third detachable connection is established.

In the context of the invention, features, which are described with regard to different embodiments of the invention and/or different categories of claims (method, use, apparatus, system, arrangement, etc.) can be combined to form further embodiments of the invention. For example, a claim relating to an apparatus can also be developed with features that are described or claimed in connection with a method and vice versa. Herein, functional features of a method can be implemented by correspondingly embodied material components. The use of the indefinite articles “a” or “an” does not preclude the relevant feature also being present multiple times.

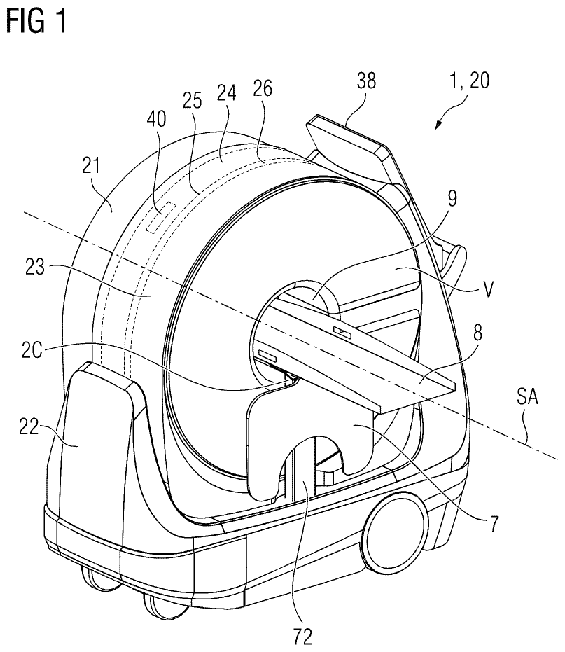

FIG. 1 shows the computed tomography device 1 in the form of a mobile computed tomography device having a gantry 20 with an opening 9 and the support structure 8,
wherein the opening 9 extends in a tunnel shape along a system axis SA of the gantry 20 in such a way that the support structure 8 and the examination object 13 can be introduced together into the opening 9 along the system axis SA when the longitudinal axis of the support structure 8 is parallel to the system axis SA and the examination object 13 lies on the bearing surface 80 between the first side wall 81 and the second side wall 82,
wherein the computed tomography device 1 has a first device-side interface 1C, 2C,
wherein the support structure 8 has a support-side interface C,
wherein the first device-side interface 1C, 2C and the support-side interface C can establish a first detachable connection, which fixes the support structure 8 relative to the gantry 20 in such a way that the longitudinal axis of the support structure 8 is parallel to the system axis SA.

The example shown provides that the gantry 20 has a first gantry part 21 and a second gantry part 22,
wherein the first gantry part 21 has a projection data acquisition system 40,
wherein the second gantry part 22 has the first device-side interface 2C,
wherein the first gantry part 21 is mounted movably relative to the second gantry part 22 in such a way that a translational movement of the first gantry part 21 relative to the second gantry part 22 can be carried out parallel to the system axis SA, while at the same time the support structure 8 rests relative to the second gantry part 22, wherein the first device-side interface 1C, 2C and the support-side interface C establish the first detachable connection, which fixes the support structure 8 relative to the gantry 20 in such a way that the longitudinal axis of the support structure 8 is parallel to the system axis SA.

The first gantry part 21 has the pivot bearing 25 and the support structure 26, wherein the rotor 24 is connected to the support structure 26 via the pivot bearing 25 and is rotatably mounted relative to the support structure 26 about the system axis SA. The rotor 24 has the projection data acquisition system 40. The projection data acquisition system 40 has the X-ray source 41 for generating the X-rays 4 and the X-ray detector 44 for detecting the X-rays 4.

The second gantry part 22 has the holding apparatus 7, the shoulder board 7 and the touch-sensitive screen 38 for operating the computed tomography device 1. The shoulder board 7 is connected to the holding apparatus 7 via a swivel apparatus and is swivel-mounted relative to the holding apparatus 7 about a swivel axis that is horizontal and perpendicular to the system axis SA.

The gantry 20 has the cladding V for delimiting an inner area of the gantry 20 from an area surrounding the gantry 20. The gantry 20 has the third gantry part 23. The third gantry part 23 has a front side of the cladding V. The front side of the cladding V surrounds a front side of the opening 9 in a ring shape. The first gantry part 21 has a rear side of the cladding V. The rear side of the cladding V surrounds a rear side of the opening 9 in a ring shape.

The first gantry part 21 is mounted movably relative to the second gantry part 22 and relative to the third gantry part 23 in such a way that the translational movement of the first gantry part 21 relative to the second gantry part 22 and the third gantry part 23, while at the same time the second gantry part 22 and the third gantry part 23 rest relative to the support structure 8, wherein the support structure 8 is introduced into the opening 9 together with the examination object 13.

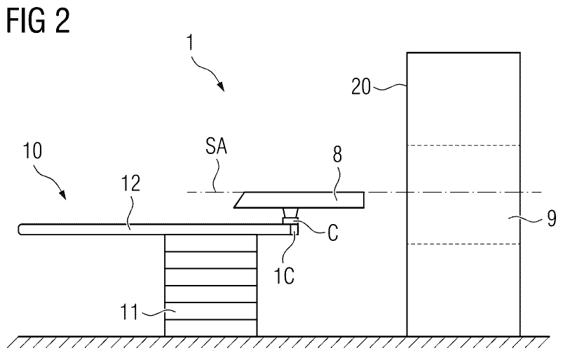

FIG. 2 shows the computed tomography device 1 with the examination table 10, wherein the examination table 10 has the first device-side interface 1C. For example, the examination table 10 can have a base 11 and a support plate 12, wherein the support plate 12 is configured to support a person, in particular an adult, in a lying position and is mounted displaceably relative to the base 11 parallel to the system axis SA of the gantry 20.

In particular, it can be provided that the first device-side interface 1C is fastened to the support plate 12 in such a way that, upon displacement of the support plate 12 relative to the base 11, the first device-side interface 1C rests relative to the support plate 12 and/or that the support structure 8 can be introduced into the opening 9 by displacing the support plate 12 relative to the base 11 when the first device-side interface 1C and the support-side interface C establish the first detachable connection, which fixes the support structure 8 relative to the gantry 20 in such a way that the longitudinal axis of the support structure 8 is parallel to the system axis SA.

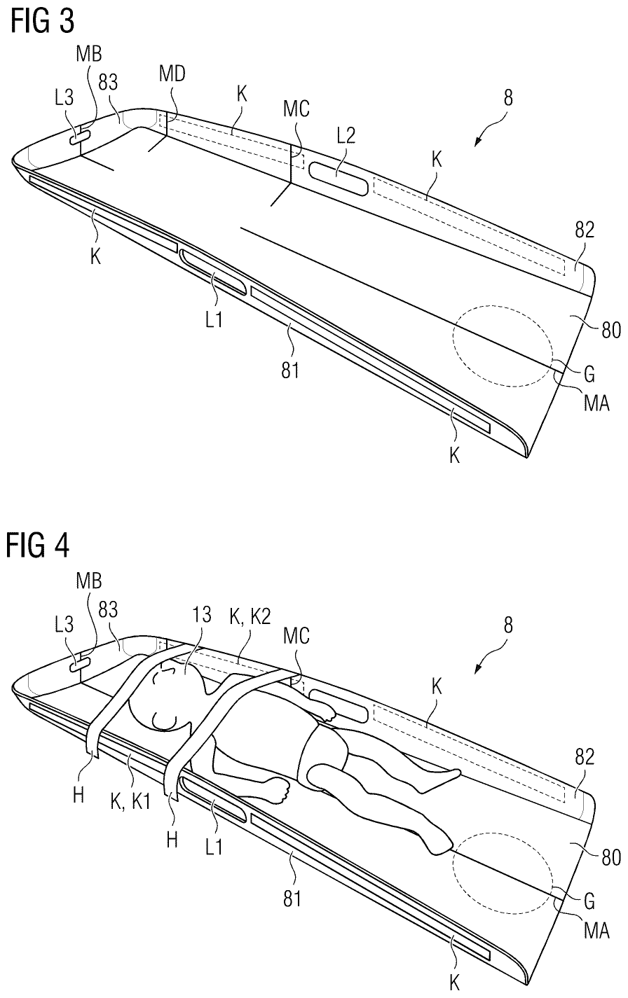

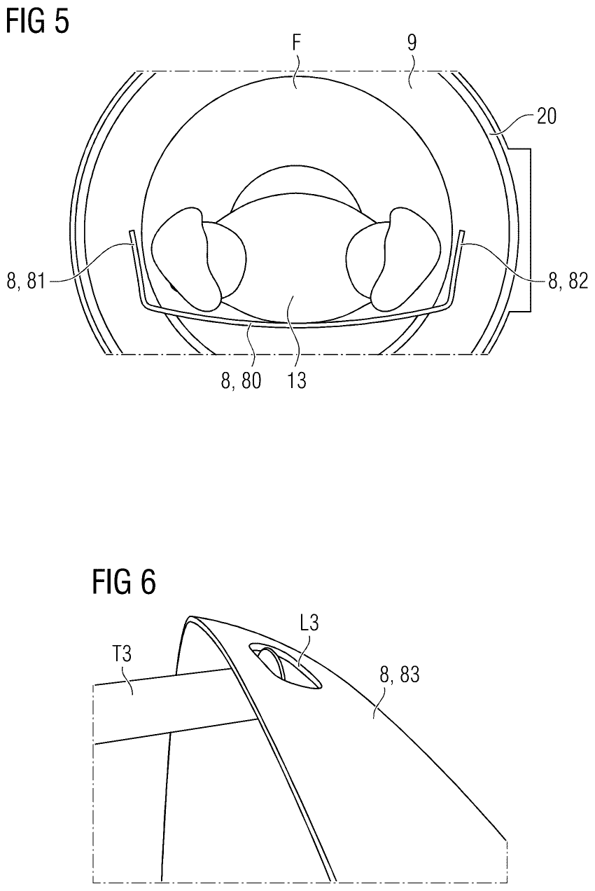

FIG. 3 to FIG. 6 show different views of the support structure 8 for supporting the examination object 13 in a pediatric computed tomography examination,
wherein the support structure 8 has a bearing surface 80 for the examination object 13, a first side wall 81 and a second side wall 82,
wherein the bearing surface 80, the first side wall 81 and the second side wall 82 in each case extend flat along a longitudinal axis of the support structure 8,
wherein the first side wall 81 and the second side wall 82 protrude relative to the bearing surface 80 in such a way that the examination object 13 can be placed on the bearing surface 80 between the first side wall 81 and the second side wall 82.

In particular, it can be provided that the first side wall 81 and the second side wall 82 protrude relative to the bearing surface 80 in such a way that, due to their planar moment of inertia, they counteract bending of the bearing surface 80 about a transverse axis of the support structure 8. In particular, it can be provided that the bearing surface 80 extends flat along a transverse axis of the support structure 8 from the first side wall 81 to the second side wall 82 and/or that the transverse axis of the support structure 8 is perpendicular to the longitudinal axis of the support structure 8.

The example shown provides that an elongated hole L1 for inserting a first palm of an operator is embodied in the first side wall 81 in such a way that the first palm can enclose an edge region of the first side wall 81 bordering this elongated hole L1 and/or
wherein an elongated hole L2 for inserting a second palm of an operator is embodied in the second side wall 82 in such a way that the second palm can enclose an edge region of the second side wall 82 bordering this elongated hole L2.

The example shown provides that the support structure 8 has a first connecting unit K1 and a second connecting unit K2 for connection to a holding strap H,
wherein the first connecting unit K1 is fastened to the first side wall 81 and the second connecting unit K2 is fastened to the second side wall 82 in such a way that the first side wall 81, the holding strap H, the second side wall 82 and the bearing surface 80 together form a circumferentially closed contour when the holding strap H is connected to the first connecting unit K1 and to the second connecting unit K2.

In particular, it can be provided that the first connecting unit K1 is fastened to the first side wall 81 in such a way that the first side wall 81 is located between the first connecting unit K1 and the examination object 13 when the examination object 13 lies on the bearing surface 80 between the first side wall 81 and the second side wall 82 and/or that the second connecting unit K2 is fastened to the second side wall 82 in such a way that the second side wall 82 is located between the second connecting unit K2 and the examination object 13 when the examination object 13 lies on the bearing surface 80 between the first side wall 81 and the second side wall 82.

The example shown provides that the support structure 8 has a rear wall 83, wherein the rear wall 83 protrudes relative to the bearing surface 80 and extends flat transversely to the longitudinal axis of the support structure 8 from the first side wall 81 to the second side wall 82. In particular, it can be provided that the rear wall 83 protrudes relative to the bearing surface 80 in such a way that, due to its planar moment of inertia, it counteracts bending of the bearing surface 80 about the longitudinal axis of the support structure 8.

The example shown provides that a hole L3 for suspending the support structure 8, in particular on a hook T3, is embodied in the rear wall 83. The example shown provides that the bearing surface 80, the first side wall 81 and the second side wall 82 merge into one another in one piece and/or in the shape of a shell. In particular, it can be provided that the bearing surface 80, the first side wall 81, the second side wall 82 and the rear wall 83 merge into one another in one piece and/or in the shape of a shell. The example shown provides that the support structure 8 has a longitudinal marking MA, which is arranged on the bearing surface 80 and extends centrally between the first side wall 81 and the second side wall 82 along the longitudinal axis of the support structure 8.

The example shown provides that the support structure 8 has a first transverse marking MC and a second transverse marking MD, which are in each case arranged on the bearing surface 80 and extend perpendicular to the longitudinal axis of the support structure 8,
wherein the first transverse marking MC and the second transverse marking MD mark an extension along the system axis SA of an examination area, which is accessible to the projection data acquisition system 40 by the translational movement of the first gantry part 21 relative to the second gantry part 22.

Velcro strips K are fastened to the side walls and form the first connecting unit K1 and the second connecting unit K2 for a velcro connection with the holding strap H.

The bearing surface 80 has a shelf tray G for medical accessories which can, for example, be connected to the examination object 13 and/or fixed relative to the support structure 8 via a holding strap. The central marking MB on the rear wall 83 extends centrally between the first side wall 81 and the second side wall 82 and is coplanar to the longitudinal marking MA.

The support structure 8 is shaped in such a way that the first side wall 81 and the second side wall 82 are located outside the acquisition area F of the projection data acquisition system 40. The acquisition area F can be understood as the field of view. This enables edges that interfere with imaging to be avoided. The bearing surface 80 is designed flat and stable such that it remains arranged relative to the acquisition area F even under load in such a way that it does not form any edges that interfere with imaging.

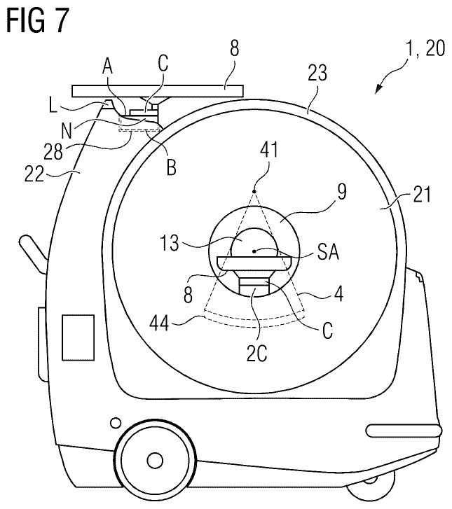

FIG. 7 shows a further view of the computed tomography device 1 in the form of a mobile computed tomography device. The example shown provides that the second gantry part 22 has a second device-side interface AC,
wherein the second device-side interface AC and the support-side interface C can establish a second detachable connection, which fixes the support structure 8 relative to the gantry 20 in such a way that the longitudinal axis of the support structure 8 is perpendicular and at an angle to the system axis SA.

The example shown provides that the second gantry part 22 has a chassis, wherein the chassis is configured to move the gantry 20 relative to a floor of an examination room parallel to a translation direction, wherein the translation direction is perpendicular to the system axis SA. In particular, it can be provided that the system axis SA is horizontal and/or that the translation direction is horizontal. In particular, it can be provided that the longitudinal axis of the support structure 8 is substantially parallel to the translation direction when the second device-side interface AC and the support-side interface C establish the second detachable connection, which fixes the support structure 8 relative to the gantry 20 in such a way that the longitudinal axis of the support structure 8 is perpendicular and at an angle to the system axis SA.

The example shown provides that the computed tomography device 1 has a holding structure N for the support structure 8,
wherein a first partial structure A of the holding structure N has the second device-side interface AC,
wherein the second gantry part 22 has a cavity 28 for accommodating the holding structure N in a form-fitting manner in such a way that the first partial structure A of the holding structure N protrudes out of the cavity 28 when the holding structure N is accommodated in the cavity 28 in a form-fitting manner.

In particular, it can be provided that a contact surface of the second partial structure BC of the holding structure N rests against a floor of the cavity 28 when the holding structure N is accommodated in the cavity 28 in a form-fitting manner.

The cavity 28 is embodied below the warning lamp L.

FIG. 7 shows the computed tomography device 1 with two support structures 8. The examination object 13 is supported on one of the two support structures 8 for an examination via the computed tomography device 1.

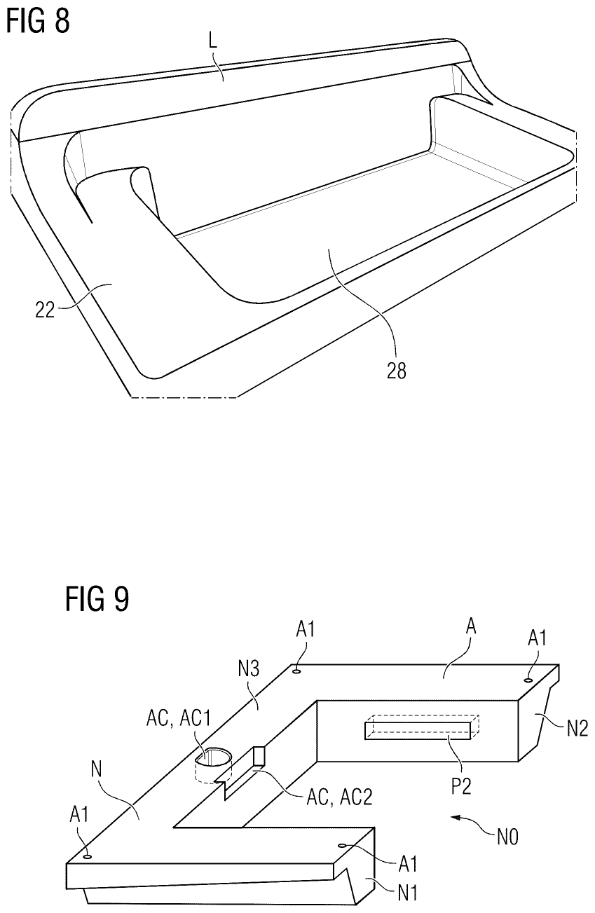

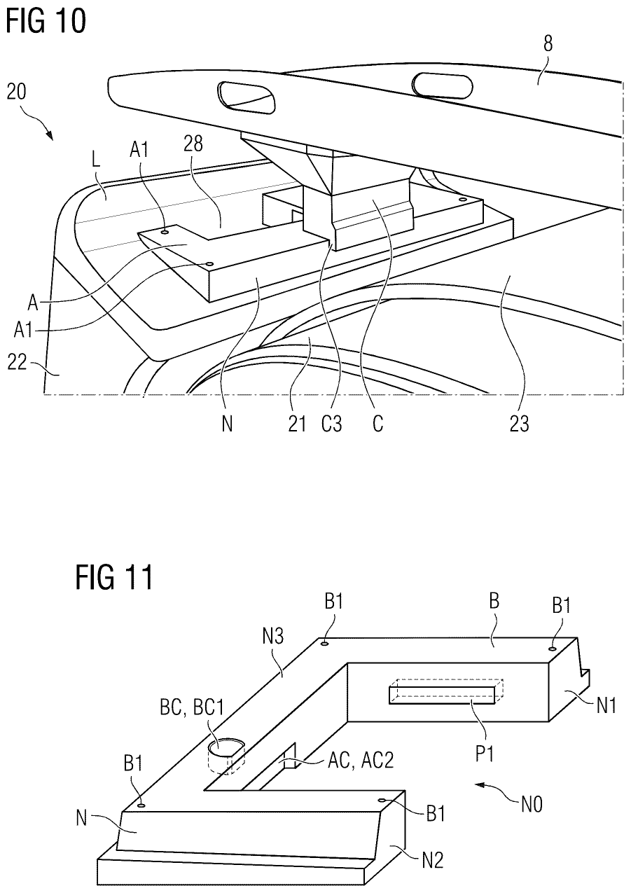

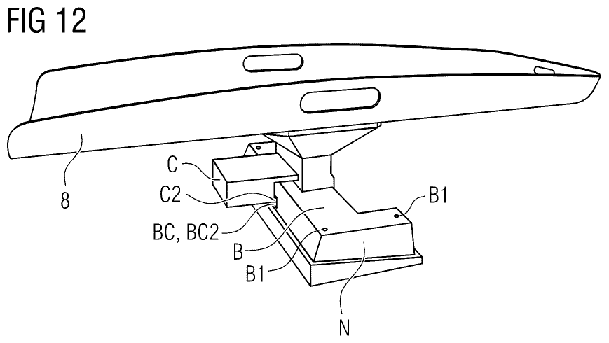

FIG. 8 shows the cavity 28 for accommodating the holding structure N in a form-fitting manner. FIG. 9 shows the holding structure N in a first orientation. FIG. 10 shows an arrangement of the support structure 8 on the gantry 20 with the aid of the holding structure N. FIG. 11 shows the holding structure N in a second orientation. FIG. 12 shows an arrangement of the support structure 8 on a table top with the aid of the holding structure N.

The example shown provides that a second partial structure B of the holding structure N has a table-side interface BC,
wherein the table-side interface BC and the support-side interface C can establish a third detachable connection, which fixes the support structure 8 relative to the holding structure N in such a way that the longitudinal axis of the support structure 8 is substantially parallel to a table top when the holding structure N is placed on the table top in such a way that a contact surface of the first partial structure A of the holding structure N rests against the table top. The contact surface of the first partial structure A of the holding structure N and the contact surface of the second partial structure B of the holding structure N can in particular be plane-parallel.

The example shown provides that the holding structure N has a first leg region N1, a second leg region N2 and a vertex region N3,
wherein the vertex region N3 has the second device-side interface AC and/or the table-side interface BC and extends from the first leg region N1 to the second leg region N2,
wherein the first leg region N1 and the second leg region N2 extend parallel to the longitudinal axis of the support structure 8 away from the vertex region N3 when the second detachable connection or the third detachable connection is established.

The holding structure N is embodied in a U-shape. The holding structure N has the recess NO between the first leg region N1 and the second leg region N2. The first leg region N1 has the recessed grip P1, which is open toward the recess NO. The second leg region N2 has the recessed grip P2, which is open toward the recess NO.

This enables the holding structure N to be gripped more securely, in order, for example, to insert it into the cavity 28 or to lift it out of the cavity 28. If, for example, labels are attached to the floor of the cavity 28, they are visible from above through the recess.

For example, it can be provided that the holding structure has at least one further second device-side interface arranged offset to the second device-side interface AC, for example closer to one of the two leg regions than to the other of the two leg regions. For example, the holding structure can have three further second device-side interfaces arranged next to one another along the vertex region.

The support structure 8 can then be fixed transversely to the longitudinal axis of the support structure 8 offset relative to the holding structure N, for example to allow easier access to the support structure 8.

In FIG. 11, the holding structure N is rotated by 180 degrees relative to the orientation of the holding structure N shown in FIG. 9. The orientation of the leg regions N1, N2 relative to the support structure 8 shown in FIG. 11 causes them to counteract tilting of the support structure 8 when the holding structure N with the support structure 8 fixed thereto is placed on a table top. If the holding structure N is accommodated in the cavity 28 in a form-fitting manner, the form fit of the holding structure N with the edges of the cavity 28 counteracts tilting. In particular, this form fit can be embodied in such a way that the holding structure N can only be removed from the floor of the cavity 28 by lifting the holding structure N vertically relative to the cavity 28.

The second device-side interface AC has the interface element AC1 and the interface element AC2. The table-side interface BC has the interface element BC1 and the interface element BC2.

The support-side interface C has the interface element C2, which corresponds to the interface elements AC2 and BC2. The support-side interface C has a further interface element corresponding to the interface elements AC1 and BC1.

The support-side interface C has the interface element C3 in the form of a protruding shoulder. The second device-side interface AC has a contact surface for a form fit with the interface element C3. This form fit supports a stable alignment of the longitudinal axis of the support structure 8 relative to the holding structure N. The first device-side interface 2C has a contact surface for a form fit with the interface element C3. This form fit supports a stable alignment of the longitudinal axis of the support structure 8 relative to the gantry 20.

The rubber buffers A1 counteract slippage of the holding structure N relative to the table top when the contact surface of the first partial structure A of the holding structure N rests against the table top. The elements B1 can, for example, be rubber buffers for use on the floor of the cavity 28 and/or access openings for mounting the rubber buffers A1.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in detail by the preferred exemplary embodiments, the invention is not restricted by the disclosed examples and other variations can be derived from the person skilled in the art without departing from the protective scope of the invention.

1. A support structure for supporting an examination object in a pediatric computed tomography examination, the support structure comprising:
a bearing surface for the examination object;
a first side wall; and
a second side wall, wherein
the bearing surface, the first side wall and the second side wall in each case extend flat along a longitudinal axis of the support structure, and
the first side wall and the second side wall protrude relative to the bearing surface such that the examination object is placeable on the bearing surface between the first side wall and the second side wall.

2. The support structure of claim 1, wherein at least one of
a first elongated hole for receiving a first palm of an operator is in the first side wall such that the first palm can enclose an edge region of the first side wall bordering the first elongated hole, or
a second elongated hole for receiving a second palm of an operator is in the second side wall such that the second palm can enclose an edge region of the second side wall bordering the second elongated hole.

3. The support structure of claim 1, further comprising:
a first connecting unit configured to connect to a holding strap, the first connecting unit fastened to the first side wall; and
a second connecting unit configured to connect to the holding strap, the second connecting unit fastened to the second side wall such that the first side wall, the holding strap, the second side wall and the bearing surface together form a circumferentially closed contour when the holding strap is connected to the first connecting unit and to the second connecting unit.

4. The support structure of claim 1, further comprising:
a rear wall, the rear wall protruding relative to the bearing surface and extending flat transversely to the longitudinal axis of the support structure from the first side wall to the second side wall.

5. The support structure of claim 4, wherein a hole for suspending the support structure is in the rear wall.

6. The support structure of claim 1, wherein the bearing surface, the first side wall and the second side wall merge into one another in at least one of one piece or a shape of a shell.

7. The support structure of claim 1, further comprising:
a longitudinal marking on the bearing surface and extending centrally between the first side wall and the second side wall along the longitudinal axis of the support structure.

8. A computed tomography device comprising:
a gantry with an opening; and
the support structure of claim 1, wherein
the opening extends in a tunnel shape along a system axis of the gantry such that the support structure and the examination object can be introduced together into the opening along the system axis when the longitudinal axis of the support structure is parallel to the system axis and the examination object lies on the bearing surface between the first side wall and the second side wall,
the computed tomography device includes a first device-side interface,
the support structure includes a support-side interface, and
the first device-side interface and the support-side interface are configured to establish a first detachable connection, the first detachable connection fixes the support structure relative to the gantry such that the longitudinal axis of the support structure is parallel to the system axis.

9. The computed tomography device of claim 8, wherein
the gantry includes a first gantry part and a second gantry part,
the first gantry part includes a projection data acquisition system,
the second gantry part includes the first device-side interface, and
the first gantry part is mounted movably relative to the second gantry part such that a translational movement of the first gantry part relative to the second gantry part can be carried out parallel to the system axis while the support structure rests relative to the second gantry part, the first device-side interface and the support-side interface establish the first detachable connection.

10. The computed tomography device of claim 9, wherein
the support structure includes a first transverse marking and a second transverse marking on the bearing surface and extend perpendicular to the longitudinal axis of the support structure, and
the first transverse marking and the second transverse marking mark an extension along the system axis of an examination area, the examination area is accessible to the projection data acquisition system by the translational movement of the first gantry part relative to the second gantry part.

11. The computed tomography device of claim 9, wherein
the second gantry part includes a second device-side interface, and
the second device-side interface and the support-side interface are configured to establish a second detachable connection, the second detachable connection fixes the support structure such relative to the gantry that the longitudinal axis of the support structure is perpendicular and at an angle to the system axis.

12. The computed tomography device of claim 11, wherein
the second gantry part includes a chassis, the chassis is configured to move the gantry relative to a floor of an examination room parallel to a translation direction, wherein the translation direction is perpendicular to the system axis.

13. The computed tomography device of claim 11, further comprising:
a holding structure for the support structure, a first partial structure of the holding structure including the second device-side interface, wherein
the second gantry part includes a cavity to accommodate the holding structure in a form-fitting manner such that the first partial structure of the holding structure protrudes out of the cavity when the holding structure is accommodated in the cavity in a form-fitting manner.

14. The computed tomography device of claim 13, wherein
a second partial structure of the holding structure includes a table-side interface,
the table-side interface and the support-side interface are configured to establish a third detachable connection, the third detachable connection fixes the support structure relative to the holding structure such that the longitudinal axis of the support structure is substantially parallel to a table top when the holding structure is on the table top such that a contact surface of the first partial structure of the holding structure rests against the table top.

15. The computed tomography device of claim 14, wherein
the holding structure includes a first leg region, a second leg region and a vertex region,
the vertex region includes at least one of the second device-side interface or the table-side interface and extends from the first leg region to the second leg region, and
the first leg region and the second leg region extend parallel to the longitudinal axis of the support structure away from the vertex region when the second detachable connection or the third detachable connection is established.

16. The support structure of claim 5, wherein the bearing surface, the first side wall and the second side wall merge into one another in at least one of one piece or a shape of a shell.

17. The support structure of claim 16, further comprising:
a longitudinal marking on the bearing surface and extending centrally between the first side wall and the second side wall along the longitudinal axis of the support structure.

18. The computed tomography device of claim 10, wherein
the second gantry part includes a second device-side interface, and
the second device-side interface and the support-side interface are configured to establish a second detachable connection, the second detachable connection fixes the support structure such relative to the gantry that the longitudinal axis of the support structure is perpendicular and at an angle to the system axis.

19. The computed tomography device of claim 18, wherein
the second gantry part includes a chassis, the chassis is configured to move the gantry relative to a floor of an examination room parallel to a translation direction, wherein the translation direction is perpendicular to the system axis.
